(19) United States
(12) Patent Application Publication
(10) Pub. No.: US 2012/0165520 A1
(43) Pub. Date: Jun. 28, 2012

(54) PROCESS FOR THE SYNTHESIS OF PRODRUGS OF OPIOIDS

(76) Inventors: Paul McGee, Baskingstoke (GB); Ian Garnett, Baskingstoke (GB); E. Juan, Baskingstoke (GB); A. Manage, Baskingston (GB); Jean-Francois Carniaux, Baskingstoke (GB)

(21) Appl. No.: 13/335,918

(22) Filed: Dec. 22, 2011

Related U.S. Application Data
(60) Provisional application No. 61/427,106, filed on Dec. 23, 2010.

(30) Foreign Application Priority Data
Jul. 4, 2011 (GB) 1111381.8

(51) Int. Cl. C07D 223/04; C07D 403/12

(52) U.S. Cl. 540/603; 540/611

(57) ABSTRACT

The present invention provides a process for the synthesis of opioid prodrugs. In particular, the present invention provides a process for the synthesis of opioid prodrugs comprising: treating an opioid, in the form of a salt or a freebase, with a carbonyl synthon to form an activated intermediate and subsequently reacting the activated intermediate with an amine, in the form of a salt or a freebase.

### CROSS REFERENCE TO RELATED APPLICATIONS

This application claims priority under 35 U.S.C. 119(e) to U.S. Provisional Application No. 61/427,106 filed Dec. 23, 2010 and also claims the benefit of GB Provisional Application No. 1111381.8 filed Jul. 4, 2011.

### FIELD OF THE INVENTION

The present invention relates to a process for the synthesis of opioid prodrugs.

### BACKGROUND OF THE INVENTION

Appropriate treatment of pain continues to represent a major challenge for both patients and healthcare professionals. Optimal pharmacologic management of pain requires selection of the appropriate analgesic drug that achieves rapid efficacy with minimal side effects. Opioid analgesics offer perhaps the most important option in the treatment of nociceptive pain and remain the gold standard of treatment.

A major shortcoming of many of the opioids is that they suffer from poor oral bioavailability due to first pass glucuronidation of the commonly present phenolic function. This has been shown, for example, with oxymorphone (Sloan et al. (2005). Supp Care Cancer 13, 57-65), meptazinol (Norbury et al. (1983). Eur J Clin Pharmacol 25, 77-80) and buprenorphine (Kintz and Marquet (2002). pp 1-11 in Buprenorphine Therapy in Opiate Addiction, Humana press). Such poor oral bioavailability results in variable blood levels of the respective opioid, and therefore, variable patient response—a highly undesirable feature in the treatment of pain where rapid and reliable relief is demanded.

Various types of prodrugs have historically been proposed to minimize first pass metabolism and so improve the oral bioavailability of opioids. These have included simple ester conjugates which are frequently hydrolyzed by plasma esterases extremely quickly. Such rapid hydrolysis by plasma esterases limits the utility of ester linked prodrugs and denies the necessary transient protection of the opioid against first past metabolism.

The rapidity of hydrolysis of ester conjugates is illustrated by work on the morphine ester prodrug morphine-3-propionate. Morphine has a poor oral bioavailability due to extensive first pass glucuronidation at the 3 and the 6 positions, resulting in much inter and intra subject variability in analgesic response after an oral dose of the drug (Hoskin (1989). Br. J. Clin Pharmacol 27, 499-505). The plasma and tissue stability of the 3-propionate prodrug is investigated, and it is found to be hydrolyzed in human plasma with a half-life of less than 5 minutes (Goth et al. (1997). International Journal of Pharmaceutics 154, 149-155).

Meptazinol is another opioid with poor oral bioavailability (<10%). The low oral bioavailability has been attributed to high first pass glucuronidation (Norbury et al. (1983) Eur. J. Clin. Pharmacol. 25, 77-80). Attempts have been made to overcome this problem by the use of ester linked meptazinol prodrugs (Lu et al. (2005). Biorg. and Med. Chem. Letters 15, 2607-2609 and Xie et al. (2005). Biorg. and Med. Chem. Letters 15, 493-4956). However, only one of these prodrugs—((Z)-3-[2-(propionyloxy)phenyl]-2-propenoic ester) showed a significant increase in bioavailability over meptazinol itself, when tested in a rat model. However, to the Applicants knowledge, no further data has been published on this prodrug.

An alternative strategy for creating a prodrug from the hydroxylic/phenolic function present in the opioids is the formation of O-alkyl (alkyl ether) or aryl ether conjugates. However, such derivatives appear to be very resistant to hydrolysis and metabolic activation. This is best illustrated by the 3-methyl ether prodrug of morphine—codeine. While codeine is not originally developed as a prodrug of morphine, it is subsequently found to give rise to small quantities of morphine. It has been estimated that less than 5% of an oral dose of codeine is converted to morphine—reflecting the slowness with which O-dealkylation takes place (Vree et al. (1992). Biopharma Drug Dispos. 13, 445-460 and Quiding et al. (1993). Eur. J. Clin. Pharmacol. 44, 319-323). The same phenomenon is observed for the corresponding dihydromorphine prodrug—dihydrocodeine, with less than 2% of an oral dose of dihydrocodeine being converted to dihydromorphine (Balikova et al. (2001). J. Chromatog. Biomed. Sci. Appl. 752, 179-186).

A further disadvantage of the O-alkyl ether prodrugging strategy is that the dealkylation of these opioids is effected by cytochrome P450 2D6 (Cyp2D6), a polymorphically expressed enzyme (Schmidt et al. (2003). Int. J. Clin. Pharmacol. Ther. 41, 95-106). This polymorphic enzyme expression inevitably results in substantial variation in patient exposure to the respective active metabolite (e.g., morphine and dihydromorphine). For example, low/negligible exposure to morphine derived from codeine has been reported amongst a large group of patients deficient in Cyp2D6 activity, potentially impacting the analgesic efficacy of codeine (Poulsen et al. (1998). Eur. Clin. Pharmacol. 54, 451-454).

An ideal prodrug moiety and linkage for a particular opioid would afford theoptimal balance of protection against first pass metabolism and susbquent efficient release of the active drug. There therefore remains a real need in the treatment of severe pain with opioids for products which retain all the inherent pharmacological advantages of the opioids, but which avoid or reduce their principal limitations of (1) low and erratic systemic availability after oral dosing and (2) induction of adverse GI side effects, including emesis and chronic constipation.

Carbamate prodrugs of opioids which feature a hydroxyl group have been shown to improve the opioid's systemic availability and/or minimize adverse gastrointestinal side-effects associated with the administration of the parent compound (PCT/GB2010/052211). The present application addresses the problem of synthesising such prodrugs.

International Application No. PCT/GB2011/052566 is herein incorporated by reference in its entirety.

### SUMMARY OF THE INVENTION

In a first aspect the present invention provides a method for synthesizing an opioid prodrug of formula (I) or a pharmaceutically acceptable salt thereof:

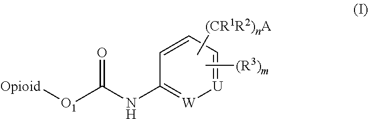

wherein:
“Opioid-O₁” is an opioid drug fragment having a phenolic hydroxyl residue and O₁ is said phenolic hydroxyl residue of the opioid;
W and U are each independently selected from the group consisting of: —CR⁴═ and —N═;
R¹ and R² are each independently selected from the group consisting of: H, hydroxy, carboxy, carboxamido, imino, alkanoyl, cyano, cyanomethyl, nitro, amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;
n iso, 1 or 2;
m is 0, 1 or 2;
R³ is independently selected from the group consisting of: hydroxy, carboxy, carboxamido, imino, alkanoyl, cyano, cyanomethyl, nitro, amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

### R⁴ is H or R³;

A is a carboxylic acid group (i.e. —CO₂H) or is a protected carboxylic acid group;
the method including the step of:
i) treating an opioid (i.e. a compound of Formula opioid-O₁—H), in the form of a salt or a freebase, with a carbonyl synthon of Formula (II),

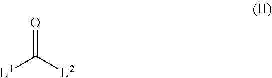

to form an activated intermediate of Formula (III)

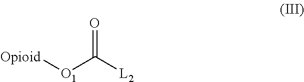

wherein:
L¹ and L² are each independently a leaving group;
the method further including the step of:
ii) reacting the activated intermediate of Formula III with an amine of Formula IV, in the form of a salt or a freebase,

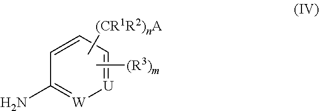

to provide the opioid prodrug of formula I.

In an embodiment, A is a carboxylic acid group or a protected carboxylic acid group selected from the group consisting of: —CO₂R⁵; —CN; —C(OR^{a})₃; —C(O)(SR⁵) and 2-oxazalinyl;

wherein R₅ is H or a protecting group;
wherein the 2-oxazalinyl group is optionally substituted with 1 or 2 substituents selected from the group consisting of: C₁-C₄ alkyl, benzyl (optionally substituted with one or two substituents selected from C₁-C₄ alkyl, C₁-C₄ alkoxy, C₁-C₄haloalkyl, C₁-C₄ haloalkoxy, halogen, CN and NO₂) and C₁-C₄haloalkyl; and
wherein R^{a} is independently at each occurrence selected from the group consisting of: C₁-C₄ alkyl and benzyl.

In an embodiment, A is a protected carboxylic acid group and the method further includes the subsequent step of reacting the opioid prodrug of Formula I to reveal the opioid prodrug of Formula I in which A is a carboxylic acid group (i.e. CO₂H).

In an embodiment, A is a protected carboxylic acid group of the formula —CO₂R₅ (wherein R⁵ is not H) and the method further includes the subsequent step of removing the protecting group (R⁵) from the opioid prodrug of Formula I to reveal the opioid prodrug of Formula I in which R⁵ is H.

In an embodiment, the method further comprises a step prior to reacting the opioid with a carbonyl synthon of Formula II including treating an acid addition salt of the opioid with a base to form the opioid freebase.

In a second aspect the present invention provides a method for synthesizing an activated opioid intermediate of Formula III:

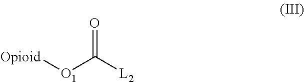

the method including the step of treating an opioid (i.e. a compound of Formula opioid-O₁—H), in the form of a salt or a freebase, with a carbonyl synthon of Formula (II),

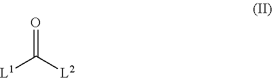

to form an activated intermediate of Formula (III)

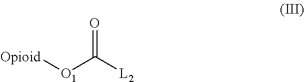

wherein:
“Opioid-O₁” is an opioid drug fragment having a phenolic hydroxyl residue and O₁ is said phenolic hydroxyl residue of the opioid;
wherein:
L¹ and L² are each independently a leaving group.

The above aspect and embodiments are illustrated in scheme 1:

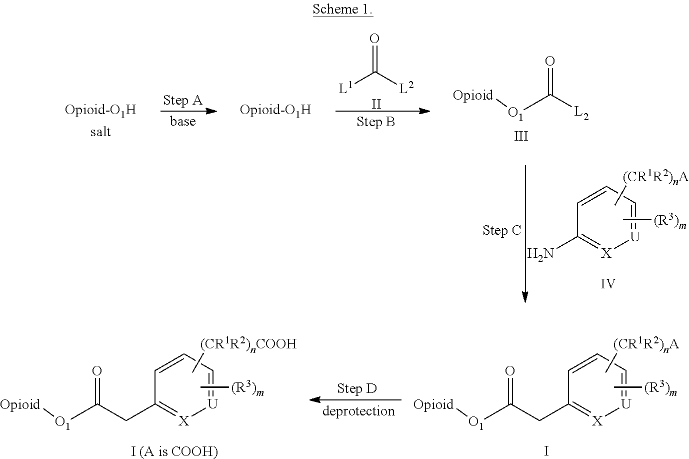

Thus the first aspect is represented in Scheme 1 as Steps B and C. The second aspect is represented in Scheme 1 as Step B.

The opioid drug fragment is covalently bonded to the rest of the prodrug at a hydroxyl group (e.g. a phenolic hydroxyl group) via a carbamate linkage. Cleavage of the carbamate linkage releases the opioid.

In an embodiment, the opioid drug having a phenolic hydroxyl group is an opioid drug selected from the group consisting of: hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated phenazepine analgesic, such as a 2-, 3- or 4-phenolically hydroxylated ethoheptazine, proheptazine, metethoheptazine or metheptazine, or any other analgesic. Alternatively the opioid may be a narcotic antagonist for example alvimopan, de-glycinated alvimopan, naloxone, N-methyl naloxone, nalorphine, naltrexone or N-methyl naltrexone.

In a preferred embodiment, the opioid drug is meptazinol.

In an alternate preferred embodiment, the opioid drug is buprenorphine.

In one embodiment, the opioid prodrug moiety is selected from one of the prodrug moieties provided in Table 1.

TABLE 1
Various prodrugs of the present invention

| | Prodrug Moiety (Amine of | |
|---|---|---|
| | Formula IV) | Structure When Bound to Opioid |
| 1 | 2-amino benzoic acid | 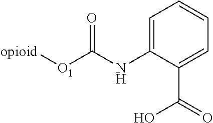 |
| 2 | 3-amino benzoic acid | 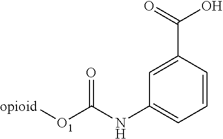 |
| 3 | 4-amino benzoic acid (PABA) | 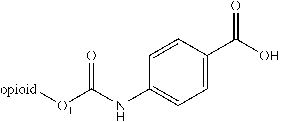 |
| 4 | 4-amino salicylic acid | 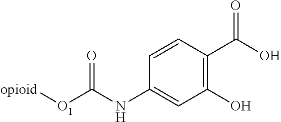 |
| 5 | 4-amino-phenyl acetic acid | 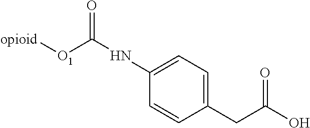 |
| 6 | 4-amino-2-chlorobenzoic acid | 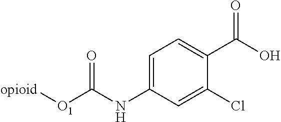 |
| 7 | 6-aminonicotinic acid | 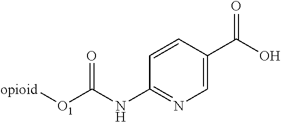 |
| 8 | 2-(4-aminophenyl) propanoic acid | 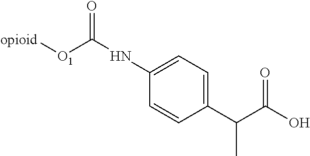 |
| 9 | 4-amino 2-fluorobenzoic acid | 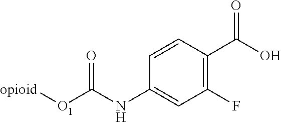 |
| 10 | 4-amino-3-ethylbenzoic acid | 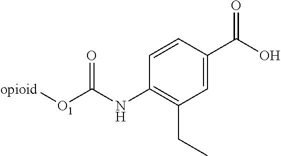 |
| 11 | 4-amino-3-methoxybenzoic acid | 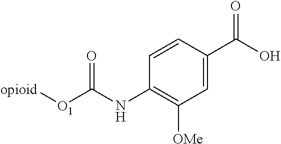 |
| 12 | 4-amino 2-methylbenzoic acid | 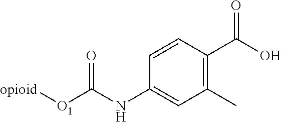 |
| 13 | 4-amino-2-methoxybenzoic acid | 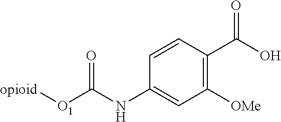 |

In a preferred embodiment, the opioid prodrug of Formula I has the structure:

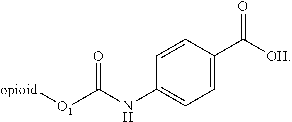

### DETAILED DESCRIPTION OF THE INVENTION

### Definitions

The term “opioid” refers to a natural (e.g. morphine), semi-synthetic (e.g. buprenorphine) or synthetic (e.g. meptazinol) drug that acts by binding to one or more of the opioid receptors in the brain, thus displacing an endogenous analgesic ligand, namely an enkephalin or endorphin, and having a therapeutically useful pain-relieving effect. “Opioid” refers to the opioid per se, as well as any active metabolites of the respective opioid.

The term “narcotic antagonist” refers to a non-natural compound which will displace an opioid from its binding site and so reverse the effects of an opioid analgesic.

The term 2-, 3- or 4-phenolically hydroxylated phenazepine analgesic means a compound having the general structure:

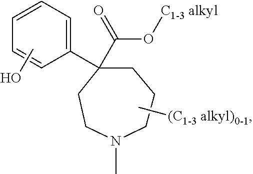

wherein each C₁₋₃ alkyl group is independently selected from the group consisting of: methyl, ethyl and n-propyl, optionally methyl and ethyl.

The term “amino” refers to a

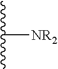

group, wherein each R is independently selected from the group consisting of: H and C₁-C₁₀ alkyl. For example, the term “amino” may refer to a

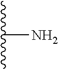

group. In the processes of this invention it may be necessary to protect an amino group with a suitable protecting group e.g. a carbamate, sulphonate, amide or benzyl protecting group. For example, an amino group may be protected using a protecting group selected from the group consisting of: tert-butyl carbonate (BOC), a benzyl carbonate (Z), fluorenylmethyl carbonate (FMOC), tosylate, mesylate, benzyl, para-methoxybenzyl, benzoyl and acetyl. Other suitable protecting groups will be readily apparent to those skilled in the art.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl, e.g. a C₁, C₂, C₃, C₄, C₅, C₆, C₇, C₅, C₉ or C₁₀ alkyl. For example, C₁₋₁₀ alkyl means a straight or branched saturated hydrocarbon chain containing, for example, at least 1, and at most 10, carbon atoms. Examples of “alkyl” groups, as used herein include, but are not limited to, methyl, ethyl, n-propyl, n-butyl, n-pentyl, isobutyl, isopropyl, t-butyl, hexyl, heptyl, octyl, nonyl and decyl.

The term “alkyl ester,” includes, for example, groups of the formulae

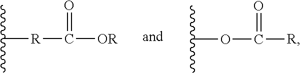

wherein each occurrence of R is independently a straight or branched C₁-C₁₀ alkyl group as defined immediately above.

The term “substituted alkyl” as used herein denotes alkyl radicals wherein at least one hydrogen is replaced by one more substituents such as, but not limited to, hydroxy, alkoxy (for example, C₁-C₁₀ alkoxy, e.g. methoxy or ethoxy), aryl (for example, phenyl), heterocycle, halogen (for example, F, Cl or Br), haloalkyl (for example, C₁-C₁₀ fluoroalkyl, e.g. trifluoromethyl or pentafluoroethyl), cyano, cyanomethyl, nitro, amino (e.g. a

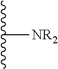

group, wherein each R is independently selected from the group consisting of: H and C₁-C₁₀ alkyl, or a

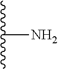

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). The definition pertains whether the term is applied to a substituent itself or to a substituent of a substituent.

The terms “amino benzoic acid analogue,” and “ABA analogue,” refer to residues having the general structure:

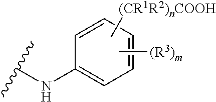

in which R¹, R², R³, n and m are as defined above.

Alternatively or additionally, an ABA analogue may have an additional substituent on the 5- or 6-membered ring (besides the acid and amino groups). For example, the ring of the ABA analogue may be further substituted with a halogen (for example, F, Cl, Br), C₁-C₆ alkyl (for example, C₁, C₂, C₃ or C₄ alkyl), C₁-C₆ alkyl ester (for example, C₁, C₂, C₃ or C₄ alkyl ester), C₁-C₆ substituted alkyl (for example, C₁, C₂, C₃ or C₄ substituted alkyl), substituted C₁-C₆ alkyl ester (for example, C₁, C₂, C₃ or C₄ substituted alkyl ester), hydroxy or amino. Alternatively or additionally, the amino group in the ABA or ABA analogue can be substituted with an alkyl or substituted alkyl group (for example, a C₁, C₂, C₃ or C₄ alkyl or substituted alkyl). Further, in contrast to ABA, an ABA analogue may have an optionally substituted C₁-C₃ n-alkyl group between the amino group (i.e., ABA's N-terminus) and the 5- or 6-membered ring.

The ABA or ABA analogue is bound to an opioid through the ABA analogue's amino group, to form a carbamate bond. In one embodiment, the ABA analogue includes a heteroaryl ring, for example a pyridine ring. In other embodiments, the ABA analogue does not include a heteroaryl ring.

The terms “para amino benzoic acid analogue,” and “PABA analogue,” refer to residues having the general structure:

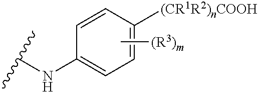

in which R¹, R², R³, n and m are as defined above.

Alternatively or additionally, a PABA analogue may have an additional substituent on the 5- or 6-membered ring (besides the acid and amino groups). For example, the ring of the PABA analogue may be further substituted with a halogen (for example, F, Cl, Br), C₁-C₆ alkyl (for example, C₁, C₂, C₃ or C₄ alkyl), C₁-C₆ alkyl ester (for example, C₁, C₂, C₃ or C₄ alkyl ester), C₁-C₆ substituted alkyl (for example, C₁, C₂, C₃ or C₄ substituted alkyl), substituted C₁-C₆ alkyl ester (for example, C₁, C₂, C₃ or C₄ substituted alkyl ester), hydroxyl or amino. Alternatively or additionally, the amino group in the PABA or PABA analogue can be substituted with an alkyl or substituted alkyl group (for example, a C₁, C₂, C₃ or C₄ alkyl or substituted alkyl). Further, in contrast to PABA, a PABA analogue may have an optionally substituted C₁-C₃ n-alkyl group between the amino group (i.e., PABA's N-terminus) and the 5- or 6-membered ring. In an embodiment, the phenyl ring of the PABA analogue is directly bonded to the amino group of the PABA analogue.

In the prodrugs produced by the present invention PABA or PABA analogue is bound to an opioid through the PABA analogue's amino group, to form a carbamate bond. In one embodiment, the PABA analogue includes a heteroaryl ring, for example a thiazole or pyridine ring. In other embodiments, the PABA analogue does not include a heteroaryl ring.

The term “cycloalkyl” group as used herein refers to a non-aromatic monocyclic hydrocarbon ring of from 3 to 8 carbon atoms. Exemplary are saturated monocyclic hydrocarbon rings having 1, 2, 3, 4, 5, 6, 7 or 8, carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as those recited in the paragraph defining the substitutents of a “substituted alkyl”. The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur. For example, a heterocyclic group may be:

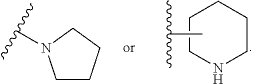

The term “substituted heterocycle” as used herein denotes a heterocycle group further bearing one or more substituents as set forth herein, such as those recited in the paragraph defining the substitutents of a “substituted alkyl”. The definition pertains whether the term is applied to a substituent itself or to a substituent of a substituent. For example, a substituted heterocyclic group may be:

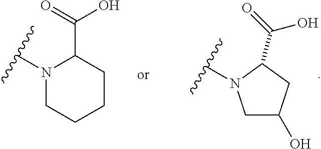

The term “aryl,” as used herein, refers to cyclic, aromatic hydrocarbon groups which have 1 to 3 aromatic rings, for example phenyl or naphthyl. The aryl group may have fused thereto a second or third ring which is a heterocyclo, cycloalkyl, or heteroaryl ring, provided in that case the point of attachment will be to the aryl portion of the ring system. Thus, exemplary aryl groups include

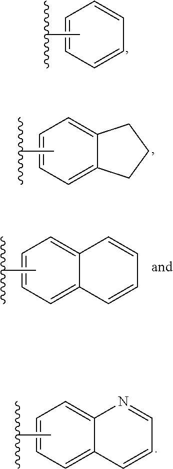

In embodiments, “aryl” refers to a ring structure consisting exclusively of hydrocarbyl groups.

The term “heteroaryl,” as used herein, refers to an aryl group in which at least one of the carbon atoms in the aromatic ring has been replaced by a heteroatom selected from oxygen, nitrogen and sulphur. The nitrogen and/or sulfur heteroatoms may optionally be oxidized and the nitrogen heteroatoms may optionally be quaternized. The heteroaryl group may be a 5 to 6 membered monocyclic, 7 to 11 membered bicyclic, or 10 to 16 membered tricyclic ring system. Thus, exemplary heteroaryl groups include

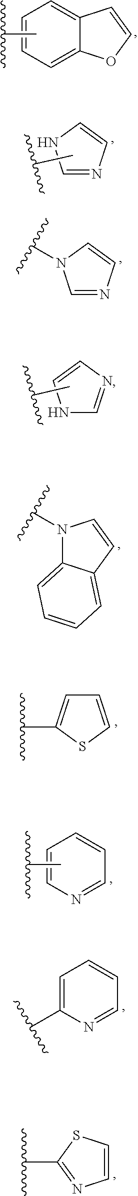

“Substituted aryl” and “substituted heteroaryl” groups refer to either an aryl or heteroaryl group, respectively, substituted by one or more substituents at any point of attachment to the aryl or heteroaryl ring (and/or any further ring fused thereto). Exemplary substituents include hydroxy, carboxyl, alkoxy (for example, C₁-C₁₀ alkoxy, e.g. methoxy, ethoxy), aryl, phenyl, heterocycle, halogen (for example F, Cl, Br), haloalkyl (for example, C₁-C₁₀ haloalkyl, e.g. trifluoromethyl or pentafluoroethyl), cyano, cyanomethyl, nitro, amino (e.g. a

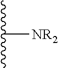

group, wherein each R is independently selected from the group consisting of: H and C₁-C₁₀ alkyl, or a

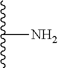

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carboxylic acid (e.g.,

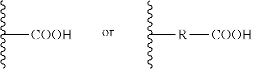

where R is a C₁-C₁₀ alkylene group such as —CH₂—), carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). For example, substituted aryl” and “substituted heteroaryl” groups include:

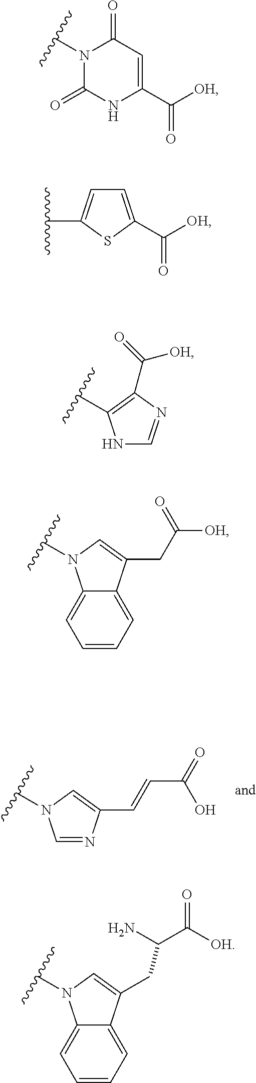

The terms “keto” and “oxo” are synonymous, and refer to the group ═O.

The term hydroxy refers to the group

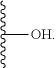

In the processes of this invention it may be necessary to protect a hydroxy group with a suitable protecting group e.g. an ester, silyl or benzyl protecting group. For example, a hydroxyl group may be protected using a protecting group selected from the group consisting of: tert-butyl diphenyl silyl (TBDPS), trialkylsilyl, acetate, benzoyl, benzyl, and substituted benzyl. Other suitable protecting groups will be readily apparent to those skilled in the art.

The terms “carbamate group,” “carbamate” and “carbamate linkage” are synonymous, and refer to the group

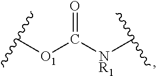

wherein the —O₁— is present in the unbound form of the opioid analgesic (e.g. the phenolic hydroxy group), and the —NR₁ moiety is an amino group present in the ABA or ABA analogue (e.g. PABA or PABA analogue). Prodrug moieties described herein may be referred to based on the ABA or ABA analogue (e.g. PABA or PABA analogue) and the carbamate linkage. The ABA or ABA analogue (e.g. PABA or PABA analogue) reference should be assumed to be bonded via an amino group present in ABA or ABA analogue (e.g. PABA or the PABA analogue) to the carbonyl linker and the opioid analgesic, unless otherwise specified.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

The term “salts” can include acid addition salts or addition salts of free bases. Suitable pharmaceutically acceptable salts (for example, of the carboxyl terminus of the PABA or PABA analogue) include, but are not limited to, metal salts for example sodium potassium and cesium salts; alkaline earth metal salts for example calcium and magnesium salts; organic amine salts for example triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts for example the hydrobromide; and organic acid salts for example trifluoroacetate salts.

### METHODS OF THE INVENTION

In an embodiment, L₁ and L₂ are independently selected from the group consisting of: halo, C₁-C₃ haloalkoxy, imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) and cyano.

In an embodiment, L₁ and L₂ are independently selected from halo and trihalo C₁-C₃ alkoxy. In another embodiment, L¹ and L² are independently selected from halo and trihalomethyloxy. In yet another embodiment, L₁ and L₂ are independently selected from Cl and OCCl₃. Thus, in an embodiment the carbonyl synthon is diphosgene (i.e. L¹ is Cl and L² is OCCl₃). In another embodiment, the carbonyl synthon is triphosgene (i.e. both L¹ and L² are OCCl₃).

In embodiments in which one or both of L₁ and L₂ are trihalomethyoxy, the L₂ group which is present in the activated intermediate of formula III is not necessarily that which was present in the carbonyl synthon of formula II. For example, in embodiments in which both L₁ and L₂ are OCX₃, wherein X is a halogen the reaction will form an activated intermediate of formula III wherein L₂ may be OCX₃ or X.

In alternative embodiments, L₁ and L₂ are imidazoles (optionally substituted with one or two substituents selected from C₁-C₄ alkyl, C₁-C₄ alkoxy, C₁-C₄haloalkyl, C₁-C₄ haloalkoxy, halogen, CN and NO₂). In an embodiment, L₁ and L₂ are both imidazole.

In an embodiment, the opioid is in the form of a salt. Alternatively, the opioid is a freebase.

### Opioids:

The opioid drug is covalently bonded to the rest of the prodrug at a hydroxyl group via a carbamate linkage.

In an embodiment, the opioid drug having a phenolic hydroxyl group is an opioid drug selected from the group consisting of: hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated phenazepine analgesic, e.g., a phenolically hydroxylated, e.g. a 2-, 3- or 4-phenolically hydroxylated of ethoheptazine, proheptazine, metethoheptazine or metheptazine, or any other analgesic. Alternatively the opioid may be a narcotic antagonist for example alvimopan, de-glycinated alvimopan, naloxone, N-methyl naloxone, nalorphine, naltrexone or N-methyl naltrexone.

In an embodiment, the opioid drug is selected from meptazinol, buprenorphine, tapentadol, nalbuphine, butorphanol, levorphanol, dextrorphan, naloxone, alvimopan and deglycinated almivopan. In another embodiment the opioid drug is selected meptazinol and buprenorphine.

In a preferred embodiment, the opioid drug is meptazinol.

In an alternate preferred embodiment, the opioid drug is buprenorphine.

### Prodrugs and Prodrug Precursors:

The following embodiments, which are independently applicable, relate to the prodrug moieties which can be coupled to opioids using the methods of the present invention. Unless otherwise stated, the following embodiments apply to prodrugs of any opioid which is described in the preceding section.

In an embodiment, A is a protected carboxylic acid group. In an embodiment, A is selected from the group consisting of: —CO₂R⁵; —CN; —C(OR^{a})₃; —C(O)(SR⁵) and 2-oxazalinyl;

wherein R⁵ is a protecting group;
wherein the 2-oxazalinyl group is optionally substituted with 1 or 2 substituents selected from the group consisting of: C₁-C₄ alkyl, benzyl (optionally substituted with one or two substituents selected from C₁-C₄ alkyl, C₁-C₄ alkoxy, C₁-C₄haloalkyl, C₁-C₄ haloalkoxy, halogen, CN and NO₂) and C₁-C₄haloalkyl;
wherein R^{a} is independently at each occurrence selected from the group consisting of: C₁-C₄ alkyl and benzyl.

In a preferred embodiment A is a protected carboxylic acid group having the formula —CO₂R⁵.

In an embodiment, n is 0.

In an alternative embodiment, n is 1 or 2.

In an embodiment, R¹ is H or methyl.

In an embodiment, R² is H or methyl.

In an embodiment, R¹ and R² are both H.

In an embodiment, R³ is selected from the group comprising: halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy) and C₁₋₆ haloalkoxy (e.g. trifluoromethoxy). In a preferred embodiment, R³ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a more preferred embodiment, R³ is selected from the group comprising: F, methyl, ethyl, methoxy and ethoxy.

In an embodiment, W is —CR⁴═, preferably R⁴ is H. In an alternative embodiment, W is —N═.

In an embodiment, U is —CR⁴═, preferably R⁴ is H.

In an embodiment, U is —CH═ and W is —CH═.

In an embodiment, m is 0.

In an alternative embodiment, m is 1.

In an embodiment, n is 0 and m is 0.

In an embodiment, n is 0 and m is 1.

In an embodiment, n is 1 and m is 0.

In an embodiment, m is 1 and R³ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, m is 1 and R³ is selected from the group comprising: F, methyl, ethyl, methoxy and ethoxy.

In an embodiment, n is 0, m is 1 and R³ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, n is 0, m is 1 and R³ is selected from the group comprising: F, methyl, ethyl, methoxy and ethoxy.

In an embodiment, U is —CH═, W is —CH═ and m is 0. In a further embodiment, U is —CH═, W is —CH═, n is 0 and m is 0.

In an embodiment, U is —CH═, W is —CH═, and m is 1. In a further embodiment, U is —CH═, W is —CH═, n is 0 and m is 1. In an embodiment, U is —CH═, W is —CH═, m is 1 and R³ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In a preferred embodiment, U is —CH═, W is —CH═, m is 1 and R³ is selected from the group comprising: F, methyl, ethyl, methoxy and ethoxy.

In an embodiment, U is —CH═, W is —CH═, n is 0, m is 1 and R³ is selected from the group comprising: halogen (e.g. fluoro, chloro or bromo), C₁₋₆ alkyl (e.g. methyl, ethyl or propyl) and C₁₋₆ alkoxy (e.g. methoxy, ethoxy or propoxy). In another embodiment, U is —CH═, W is —CH═, n is 0, m is 1 and R³ is selected from the group comprising: F, methyl, ethyl, methoxy and ethoxy.

In an embodiment, U is —CH═, W is —N═ and m is 0. In another embodiment, U is —CH═, W is —N═, n is 0 and m is 0.

In an embodiment, the opioid prodrug of Formula I has the structure:

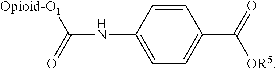

In an embodiment, the opioid prodrug of Formula I has the structure:

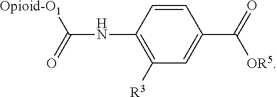

In an embodiment, the opioid prodrug of Formula I has the structure:

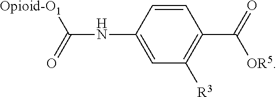

In an embodiment, the opioid prodrug of Formula I has the structure:

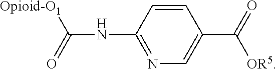

In an embodiment, the opioid prodrug of Formula I has the structure:

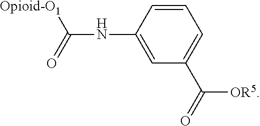

In an embodiment, the opioid prodrug of Formula I has the structure:

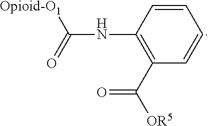

In an embodiment, R⁵ is H. In an embodiment, R⁵ is a protecting group. In an embodiment, R⁵ is the protecting group is selected from the group consisting of: C₁-C₆ alkyl, aryl C₁-C₆ alkyl, silyl (wherein the silicon is substituted with 3 groups selected from C₁-C₄ alkyl and phenyl), and heteroaryl C₁-C₆ alkyl. In an embodiment, R⁵ is the protecting group is selected from the group consisting of: C₁-C₆ alkyl, aryl C₁-C₆ alkyl and heteroaryl C₁-C₆ alkyl. In an embodiment, R⁵ is selected from the group consisting of: —CH₂-aryl (e.g. benzyl), —CH₂-substituted aryl (e.g. substituted benzyl) and —CH₂-heteroaryl. In an embodiment, R⁵ is C₁-C₆ alkyl. In an embodiment, R⁵ is —CH₂-aryl. In another embodiment, R⁵ is benzyl or tert-butyl. In a preferred embodiment, R⁵ is benzyl. In a further preferred embodiment, R⁵ is tert-butyl.

In one embodiment, the opioid prodrug moiety is selected from one of the prodrug moieties provided in Table 2.

TABLE 2
Various prodrugs of the present invention

| | Prodrug |
|---|---|
| 14 | 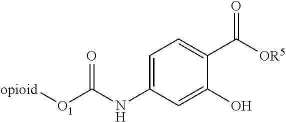 |
| 15 | 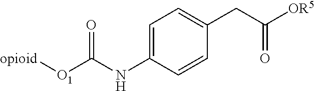 |
| 16 | 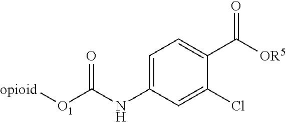 |
| 17 | 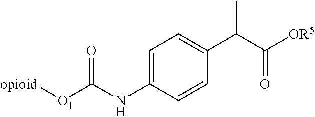 |
| 18 | 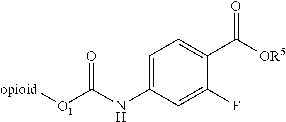 |
| 19 | 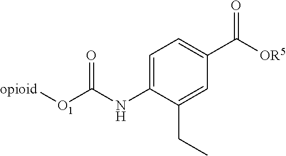 |
| 20 | 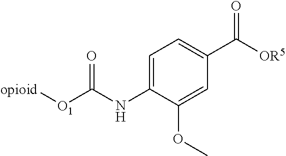 |
| 21 | 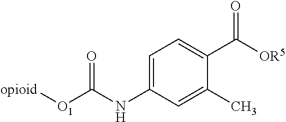 |
| 22 | 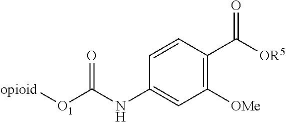 |

### Reaction Conditions:

The following embodiments, which are independently applicable, describe the reaction conditions which can be employed when performing the methods of the present invention. The following embodiments describe reaction conditions which can, unless otherwise stated, be used for the formation of any of the opioid prodrugs of formula I described in the preceding sections.

Step B (as Shown in Scheme 1):

As described above the methods of the present invention include the step of treating an opioid (i.e. a compound of Formula opioid-O₁—H), in the form of a salt or a freebase, with a carbonyl synthon of Formula II, to form an activated intermediate of Formula III (Step B).

In some embodiments, Step B is performed in the presence of a base. In an embodiment, that base is an organic base. For example, the base is selected from the group consisting of: pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) and any trialkylamine (optionally wherein two of the alkyl groups form a 5-7 membered saturated ring, optionally wherein the ring contains oxygen or nitrogen, e.g. N-methylmorpholine). The trialkylamine may be triethylamine or diisopropylethylamine. In an alternate embodiment, the base is pyridine.

In an alternative embodiment, the base is an inorganic base. In an embodiment, the base is a hydroxide, carbonate or bicarbonate of a Group 1 or Group 2 metal. In an embodiment, the base is a carbonate or bicarbonate of a Group 1 or Group 2 metal. In an embodiment, the base is selected from the group consisting of: potassium carbonate, sodium carbonate, potassium bicarbonate and sodium bicarbonate. In an embodiment, the base is in the form of an aqueous solution. In an embodiment, the base is in the form of a saturated aqueous solution. In an embodiment the base is sodium bicarbonate. In an embodiment, the sodium bicarbonate is in the form of an aqueous solution, optionally the sodium bicarbonate is in the form of a saturated aqueous solution.

In some alternative embodiments, Step B is not performed in the presence of a base.

In an embodiment, Step B is performed in a non-nucleophillic solvent. In an embodiment, Step B is performed in a solvent selected from: hexane, heptane, cyclohexane, DCM, dichloroethane, benzene, toluene and chlorobenzene. In an embodiment, Step B is performed in a solvent selected from: DCM, dichloroethane, benzene, toluene and chlorobenzene. In an embodiment, Step B is performed in DCM.

In an embodiment, Step B is performed at a temperature of from −50° C. to 70° C. In some embodiments, Step B is performed at a temperature of from −20° C. to 10° C. In yet other embodiments, Step B is performed at a temperature of from −15° C. to 0° C. In yet other embodiments, Step B is performed at a temperature of from −12° C. to −7° C.

In alternative embodiments, Step B is performed at a temperature of from 0° C. to 50° C. In other alternative embodiments, Step B is performed at a temperature of from 10° C. to 30° C. In further alternative embodiments, Step B is performed at a temperature of from 18° C. to 23° C.

In some embodiments, L₁ and L₂ are independently selected from Cl and OCCl₃ and Step B is performed in the presence of a base. In an embodiment, that base is an organic base. For example, the base could be pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) or any trialkylamine (optionally wherein two of the alkyl groups form a 5-7 membered saturated ring, optionally wherein the ring contains oxygen or nitrogen, e.g. N-methylmorpholine). The trialkylamine may be triethylamine or diisopropylethylamine. In an embodiment, the base is pyridine.

In an embodiment, L₁ and L₂ are independently selected from Cl and OCCl₃ and Step B is performed in a solvent selected from: DCM, dichloroethane, benzene, toluene, chlorobenzene. In an embodiment, L₁ and L₂ are independently selected from Cl and OCCl₃ and Step B is performed in DCM.

In an embodiment, L₁ and L₂ are independently selected from Cl and OCCl₃ and Step B is performed at a temperature of from −50° C. to 50° C. In another embodiment, L₁ and L₂ are independently selected from Cl and OCCl₃ and Step B is performed at a temperature of from −20° C. to 10° C. In yet another embodiment, L₁ and L₂ are independently selected from Cl and OCCl₃ and Step B is performed at a temperature of from −15° C. to 0° C. In yet another embodiment, L₁ and L₂ are independently selected from Cl and OCCl₃ and Step B is performed at a temperature of from −12° C. to −7° C.

In an embodiment, L₁ and L₂ are independently selected from Cl and OCCl₃ and Step B is performed in DCM in the presence of a base. In another embodiment, L₁ and L₂ are independently selected from Cl and OCCl₃ and Step B is performed in DCM in the presence of a base selected from pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) or any trialkylamine (optionally wherein two of the alkyl groups form a 5-7 membered saturated ring, optionally wherein the ring contains oxygen or nitrogen, e.g. N-methylmorpholine). In yet another embodiment, L₁ and L₂ are independently selected from Cl and OCCl₃ and Step B is performed in DCM in the presence of pyridine.

In an embodiment, L₁ and L₂ are independently selected from Cl and OCCl₃ and Step B is performed in DCM in the presence of a base at a temperature of from −15° C. to 0° C. In another embodiment, L₁ and L₂ are independently selected from Cl and OCCl₃ and Step B is performed in DCM in the presence of a base selected from pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) or any trialkylamine (optionally wherein two of the alkyl groups form a 5-7 membered saturated ring, optionally wherein the ring contains oxygen or nitrogen, e.g. N-methylmorpholine) at a temperature of from −15° C. to 0° C. In yet another embodiment, L₁ and L₂ are independently selected from Cl and OCCl₃ and Step B is performed in DCM in the presence of pyridine at a temperature of from −15° C. to 0° C.

In an embodiment, L₁ and L₂ are independently selected from Cl and OCCl₃ and Step B is performed in DCM in the presence of a base at a temperature of from −12° C. to −7° C. In another embodiment, L₁ and L₂ are independently selected from Cl and OCCl₃ and Step B is performed in DCM in the presence of a base selected from pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) or any trialkylamine (optionally wherein two of the alkyl groups form a 5-7 membered saturated ring, optionally wherein the ring contains oxygen or nitrogen, e.g. N-methylmorpholine) at a temperature of from −12° C. to −7° C. In yet another embodiment, L₁ and L₂ are independently selected from Cl and OCCl₃ and Step B is performed in DCM in the presence of pyridine at a temperature of from −12° C. to −7° C.

In an embodiment, L₁ and L₂ are imidazole and Step B is performed in a solvent selected from: DCM, dichloroethane, benzene, toluene and chlorobenzene. In an embodiment, L₁ and L₂ are imidazole and Step B is performed in DCM.

In an embodiment, L₁ and L₂ are imidazole and Step B is performed in the absence of a base.

In an embodiment, L₁ and L₂ are imidazole and Step B is performed at a temperature of from 0° C. to 50° C. In another embodiment, L₁ and L₂ are imidazole and Step B is performed at a temperature of from 10° C. to 30° C. In a further embodiment, L₁ and L₂ are imidazole and Step B is performed at a temperature of from 18° C. to 23° C.

In an embodiment, L₁ and L₂ are imidazole and Step B is performed in DCM at a temperature of from 0° C. to 50° C. In another embodiment, L₁ and L₂ are imidazole and Step B is performed in DCM at a temperature from of 10° C. to 30° C. In a further embodiment, L₁ and L₂ are imidazole and Step B is performed in DCM at a temperature of from 18° C. to 23° C.

In an embodiment, L₁ and L₂ are imidazole and Step B is performed in DCM in the absence of a base at a temperature of from 0° C. to 50° C. In another embodiment, L₁ and L₂ are imidazole and Step B is performed in DCM in the absence of a base at a temperature of from 10° C. to 30° C. In a further embodiment, L₁ and L₂ are imidazole and Step B is performed in the absence of a base in DCM at a temperature of from 18° C. to 23° C.

Step C (as Shown in Scheme 1):

As described above the methods of the present invention include the step of reacting the activated intermediate of Formula III with an amine of Formula IV, as a salt or as a freebase, to provide the opioid prodrug of formula I (Step C).

In some embodiments, Step C is performed in the presence of a base. In an embodiment, that base is an organic base. For example, the base is selected from the group consisting of: pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) and any trialkylamine (optionally wherein two of the alkyl groups form a 5-7 membered saturated ring, optionally wherein the ring contains oxygen or nitrogen, e.g. N-methylmorpholine). The trialkylamine may be triethylamine or diisopropylethylamine. In an alternate embodiment, the base is pyridine.

In an alternative embodiment, the base is an inorganic base. In an embodiment, the base is a hydroxide, carbonate or bicarbonate of a Group 1 or Group 2 metal. In an embodiment, the base is a carbonate or bicarbonate of a Group 1 or Group 2 metal. In an embodiment, the base is selected from the group consisting of: potassium carbonate, sodium carbonate, potassium bicarbonate and sodium bicarbonate. In an embodiment, the base is in the form of an aqueous solution. In an embodiment, the base is in the form of a saturated aqueous solution. In an embodiment the base is sodium bicarbonate. In an embodiment, the sodium bicarbonate is in the form of an aqueous solution, optionally the sodium bicarbonate is in the form of a saturated aqueous solution.

In an alternative embodiment, Step C is performed in the presence of an acid. The acid may be selected from HCl, TFA, acetic acid, formic acid, propionic acid, pyridinium para-toluene sulphonate, para-toluene sulfonic acid, and camphor sulfonic acid. In another embodiment, the acid is HCl. In a further alternative embodiment, Step C is performed in the presence of TFA.

In an embodiment, Step C is performed in a solvent selected from: DCM, toluene, chlorobenzene, benzene, diethyl ether, ethyl acetate, MeCN, acetic acid, formic acid, NMP, DMF, THF, TBME, DMSO, dioxane, pyridine, hexane, dichloroethane, xylene and acetonitrile or a combination of two or more of said solvents.

In an embodiment, Step C is performed in a solvent selected from: DCM, dichloroethane, benzene, toluene, chlorobenzene. In an embodiment, Step C is performed in DCM.

In an alternative embodiment, Step C is performed in a solvent selected from: NMP, MeCN, THF, diethyl ether, dioxane, acetic acid or formic acid. In another alternative embodiment Step C is performed in a solvent selected from: NMP, MeCN, THF, diethyl ether or dioxane. In a further alternative embodiment, Step C is performed in THF. In a further alternative embodiment, Step C is performed in NMP. In yet another alternative embodiment, Step C is performed in acetic acid or formic acid.

In an embodiment, the amine of formula IV is in the form of a freebase.

In an alternative embodiment, the amine of formula IV is in the form of a salt. In a further alternative embodiment, the amine of formula III is in the form of a HCl salt.

In an embodiment, Step C is performed at a temperature of from −50° C. to 80° C. In another embodiment, Step C is performed at a temperature of from −20° C. to 20° C. In yet another embodiment, Step C is performed at a temperature of from −10° C. to 10° C. In yet another embodiment, Step C is performed at a temperature of from −2° C. to 3° C.

In some alternative embodiments, Step C is performed at a temperature of from 20° C. to 60° C. In other alternative embodiments, Step C is performed at a temperature of from 30° C. to 50° C. In yet other alternative embodiments, Step C is performed at a temperature of from 35° C. to 40° C.

In an embodiment, L₂ is Cl or OCCl₃ and Step C is performed in DCM. In another embodiment, L₂ is Cl or OCCl₃ and Step C is performed in DCM in the presence of a base. In a further embodiment, L₂ is Cl or OCCl₃ and Step C is performed in DCM in the presence of a base selected from pyridine (optionally substituted with one or two substituents selected from C₁-C₄ alkyl, C₁-C₄ alkoxy, C₁-C₄haloalkyl, C₁-C₄ haloalkoxy, halogen, CN and NO₂) and any trialkylamine. In yet another embodiment, L₂ is Cl or OCCl₃ and Step C is performed in DCM in the presence of pyridine. In these embodiments, the amine of formula IV may be in the form of a freebase.

In an embodiment, L₂ is Cl or OCCl₃ and Step C is performed in DCM in the presence of a base at a temperature of from −10° C. to 10° C. In another embodiment, L₂ is Cl or OCCl₃ and Step C is performed in DCM in the presence of a base selected from pyridine (optionally substituted with one or two substituents selected from C₁-C₄ alkyl, C₁-C₄ alkoxy, C₁-C₄haloalkyl, C₁-C₄ haloalkoxy, halogen, CN and NO₂) and any trialkylamine at a temperature of from −10° C. to 10° C. In yet another embodiment, L₂ is Cl or OCCl₃ and Step C is performed in DCM in the presence of pyridine at a temperature of from −10° C. to 10° C. In these embodiments, the amine of formula IV may be in the form of a freebase.

In an embodiment, L₂ is Cl or OCCl₃ and Step C is performed in DCM in the presence of a base at a temperature of from −2° C. to 3° C. In another embodiment, L₂ is Cl or OCCl₃ and Step C is performed in DCM in the presence of a base selected from pyridine (optionally substituted with one or two substituents selected from C₁-C₄ alkyl, C₁-C₄ alkoxy, C₁-C₄haloalkyl, C₁-C₄ haloalkoxy, halogen, CN and NO₂) and any trialkylamine at a temperature of from −2° C. to 3° C. In yet another embodiment, L₂ is Cl or OCCl₃ and Step C is performed in DCM in the presence of pyridine at a temperature of from −2° C. to 3° C. In these embodiments, the amine of formula IV may be in the form of a freebase.

In an alternative embodiment, L₂ is imidazole and Step C is performed in THF. In an alternative embodiment, L₂ is imidazole and Step C is performed in THF in the presence of an acid. In an alternative embodiment, L₂ is imidazole and Step C is performed in THF in the presence of TFA. In these embodiments, the amine of formula IV may be in the form of a salt. That salt may be the HCl salt.

In an embodiment, L₂ is imidazole and Step C is performed in THF at a temperature of from 30° C. to 50° C. In an alternative embodiment, L₂ is imidazole and Step C is performed in THF in the presence of an acid at a temperature of from 30° C. to 50° C. In an alternative embodiment, L₂ is imidazole and Step C is performed in THF in the presence of TFA at a temperature of from 30° C. to 50° C. In these embodiments, the amine of formula IV may be in the form of a salt. That salt may be the HCl salt.

In an alternative embodiment, L₂ is imidazole and Step C is performed in THF at a temperature of from 35° C. to 40° C. In an alternative embodiment, L₂ is imidazole and Step C is performed in THF in the presence of an acid at a temperature of from 35° C. to 40° C. In an alternative embodiment, L₂ is imidazole and Step C is performed in THF in the presence of TFA at a temperature of from 35° C. to 40° C. In these embodiments, the amine of formula IV may be in the form of a salt. That salt may be the HCl salt at a temperature of from 35° C. to 40° C.

In an embodiment, the activated intermediate of formula III is not isolated after Step B.

In an embodiment, the activated intermediate of formula III is not separated from any base, salts or side products which may be present. In a further embodiment, the reaction mixture resulting from Step B, is concentrated in vacuo after the reaction. In yet another embodiment, the reaction mixture from Step B, is concentrated in vacuo after the reaction and then the solvent for Step C is added to the concentrated reaction mixture. These embodiments apply particularly to embodiments in which L₂ is Cl or OCCl₃.

In an alternative embodiment, the activated intermediate of formula III is separated from any base, salts or side products. In embodiments in which the activated intermediate of formula III is separated from any base and salts, the reaction mixture from Step B may be washed with water following the reaction. These features apply particularly to embodiments in which Step C is performed in the presence of an acid. These features also apply particularly to embodiments in which L₁ and L₂ are imidazole.

In embodiments in which both Step B and Step C are performed in the presence of a base, the base used in steps B and C may be the same. In an embodiment, that base is an organic base. For example, the base is selected from the group consisting of: pyridine (optionally substituted with one or two substituents selected from C₁-C₄ alkyl, C₁-C₄ alkoxy, C₁-C₄haloalkyl, C₁-C₄ haloalkoxy, halogen, CN and NO₂, e.g. 2,6-lutidine), imidazole (optionally substituted with one or two substituents selected from C₁-C₄ alkyl, C₁-C₄ alkoxy, C₁-C₄haloalkyl, C₁-C₄ haloalkoxy, halogen, CN and NO₂) and any trialkylamine (optionally wherein two of the alkyl groups form a 5-7 membered saturated ring, optionally wherein the ring contains oxygen or nitrogen, e.g. N-methylmorpholine). The trialkylamine may be triethylamine or diisopropylethylamine. In an alternate embodiment, the base is pyridine.

In an alternative embodiment, the base is an inorganic base. In an embodiment, the base is a hydroxide, carbonate or bicarbonate of a Group 1 or Group 2 metal. In an embodiment, the base is a carbonate or bicarbonate of a Group 1 or Group 2 metal. In an embodiment, the base is selected from the group consisting of: potassium carbonate, sodium carbonate, potassium bicarbonate and sodium bicarbonate. In an embodiment, the base is in the form of an aqueous solution. In an embodiment, the base is in the form of a saturated aqueous solution. In an embodiment the base is sodium bicarbonate. In an embodiment, the sodium bicarbonate is in the form of an aqueous solution, optionally the sodium bicarbonate is in the form of a saturated aqueous solution.

In an embodiment, the base used in both steps B and C is a nitrogen base. For example, the base used in both steps B and C is selected from pyridine (including substituted pyridines) or any trialkylamine. The trialkylamine may be triethylamine or diisopropylethylamine. In an embodiment, the base used in both steps B and C is pyridine.

In an embodiment, steps B and C are performed the same solvent. In an embodiment, that solvent is selected from: DCM, dichloroethane, benzene, toluene, chlorobenzene. In an embodiment, steps B and C are performed in DCM.

In an embodiment, Step C is performed in a mixture of solvents. In another embodiment, Step C is performed in a mixture of the solvent for Step C with a small amount (<5% by volume) of the solvent from Step B. In a further embodiment, Step C is performed in a mixture of THF with a small amount (<5% by volume) of DCM.

In an embodiment, L₂ is imidazole and R⁵ is H.

Step A (as Shown in Scheme 1):

In some embodiments, the method includes the step of treating a salt of the opioid with a base to form the opioid freebase (Step A).

In an embodiment, Step A is performed in a solvent selected from methanol, ethanol, isopranol, water, DCM, toluene, chlorobenzene, benzene, diethyl ether, ethyl acetate, NMP, DMF, THF, TBME, DMSO, dioxane, pyridine, hexane, dichloroethane, xylene and acetonitrile or a combination of two or more of said solvents. In another embodiment, Step A is performed in a solvent selected from water, DCM, THF or a combination of two or more of said solvents. In an embodiment, the solvent is THF. In an alternative, embodiment the solvent is water. In yet another alternative embodiment, the solvent is a combination of water and DCM.

In an embodiment the base in Step A, is an inorganic base. In an embodiment, the base is a carbonate or a bicarbonate of a Group 1 or Group 2 metal. In an embodiment, the base in Step A is selected from potassium carbonate, sodium carbonate, potassium bicarbonate and sodium bicarbonate. In a preferred embodiment, the base is sodium bicarbonate. In an alternate embodiment, the base is ammonia. If the base is ammonia and water is present the base may be ammonium hydroxide.

In an embodiment, Step A is performed in a mixture of DCM and water and the base is selected from potassium carbonate, sodium carbonate, potassium bicarbonate and sodium bicarbonate. In a preferred embodiment, Step A is performed in a mixture of DCM and water and the base is sodium bicarbonate.

In a preferred embodiment, Step A is performed in water and the base is ammonia (which forms ammonium hydroxide).

Step D (as Shown in Scheme 1):

In some embodiments, the method comprises removing the protecting group (R⁵) from an opioid prodrug of formula I in which R⁵ is not H, to generate the opioid prodrug of formula I in which R⁵ is H (Step D).

As has been described, in some embodiments R⁵ may be benzyl or substituted benzyl. In an embodiment, Step D comprises treating the opioid prodrug of formula I with an oxidant. In another embodiment, Step D comprises treating the opioid prodrug of formula I with a reductant. In an embodiment, when R⁵ is benzyl or substituted benzyl, Step D is conducted under neutral conditions.

Oxidants which may be used in Step D include ceric ammonium nitrate (CAN) and 2,3-dichloro-5,6-dicyano-1,4-benzoquinone (DDQ).

Reductants which may be used in Step D include hydrogen. In an embodiment, the reductant used in Step D is hydrogen and the reaction is performed in the presence of a hydrogenation catalyst. In an embodiment, the hydrogenation catalyst is a transition metal or a compound containing a transition metal. In an embodiment that transition metal is selected from palladium, platinum and nickel. In another embodiment the transition metal is on a support (e.g. carbon, aluminium oxide). The transition metal may be in any oxidation state. In a further embodiment the hydrogenation catalyst is selected from the group consisting of: Raney nickel, palladium on carbon, PdS, palladium on aluminium oxide, platinum on aluminium oxide, palladium oxide, palladium hydroxide, palladium black and platinum on carbon. In an embodiment, the hydrogenation catalyst is palladium on carbon. In an embodiment, the reductant in Step D is hydrogen, the reaction is performed in the presence of a hydrogenation catalyst and the reaction is performed in a solvent selected from the group consisting of: ethanol, methanol, ethyl acetate, diethyl ether, dioxane, TBME and THF. In an embodiment, Step D is performed in ethanol. In an alternate embodiment, Step D is performed in THF. In an embodiment, the hydrogenation catalyst is palladium on carbon.

In an embodiment, R⁵ is benzyl and Step D is performed in ethanol, with hydrogen as the reductant in the presence of a hydrogenation catalyst. In an alternative embodiment, R⁵ is benzyl and Step D is performed in THF with hydrogen as the reductant in the presence of a hydrogenation catalyst. In these embodiments, the hydrogenation catalyst is preferably palladium on carbon.

In an embodiment, Step C further involves the use of a metal scavenger to reduce the level of metal in the end product. In an embodiment, the reductant in Step C is hydrogen, the reaction is performed in the presence of palladium on carbon as the hydrogenation catalyst and Step C further involves the use of a palladium scavenger to reduce the level of palladium in the end product. In an embodiment, the palladium scavenger is selected from the group consisting of: Deloxan, MP-TMT, Phosphonics SPM 32 and Quadrapure-TU. In an embodiment, the palladium scavenger is Quadrapure-TU. In an embodiment, the amount of scavenger employed in Step C is from 0.8 to 1.2 wt equivalents. In an embodiment, the scavenger is contacted with the reaction mixture at a temperature of 30° C. or less, optionally at 20° C. In an embodiment, the scavenger is contacted with the reaction mixture for a period of up to 40 hours, optionally 20 hours. In an embodiment, the palladium scavenger is added to the reaction mixture after filtering the reaction mixture.

In embodiments in which the reductant is hydrogen, hydrogen may be introduced to the reaction chamber as a gas. Alternatively hydrogen may be generated in situ in a transfer hydrogenation process (using, for example, ammonium formate or cyclohexene as a source of hydrogen).

In an embodiment step D comprises treating the opioid prodrug of formula I with TMSI.

As has been described, in some embodiments R⁵ may be tert-butyl. In an embodiment, Step D comprises treating the opioid prodrug of formula I with an acid.

In an embodiment, Step D is performed using an acid selected from the group consisting of: TFA, HCl, HBr, benzenesulfonic acid, methansulfonic acid, pyridinium para-toluene sulphonate, para-toluene sulfonic acid, and camphor sulfonic acid. In an embodiment, the acid is selected from the group consisting of: TFA and HCl. In an embodiment the acid is TFA. In a further embodiment the acid is HCl. In embodiments in which the acid is HCl, the HCl may be used as an aqueous solution, as a gas or may be prepared in situ using e.g. acetyl chloride and formic acid; acetyl chloride and an alcohol (e.g. methanol or ethanol)l; or thionyl chloride and alcohol (e.g. methanol or ethanol). In an embodiment, HCl is prepared in situ using acetyl chloride and formic acid. In embodiments in which HCl is prepared in situ, water may also be present. In an embodiment, Step D is performed using an acid, and Step D is performed in a solvent selected from the group consisting of: water, DCM, toluene, chlorobenzene, benzene, diethyl ether, ethyl acetate, NMP, DMF, THF, TBME, DMSO, dioxane, pyridine, hexane, dichloroethane, xylene and acetonitrile or a combination of two or more of said solvents. In other embodiments, Step D is performed in a solvent selected from the group consisting of: DCM, dichloroethane and chlorobenzene. In alternative embodiments, no solvent is present and optionally the reagents are used in high volumes.

In an embodiment, Step D is performed using TFA in a solvent selected from a group consisting of: DCM, dichloroethane and chlorobenzene. In another embodiment Step D is performed using TFA in DCM.

In an embodiment, Step D is performed using HCl which is generated in situ using e.g. acetyl chloride and formic acid; acetyl chloride and an alcohol (e.g. methanol or ethanol)l; or thionyl chloride and alcohol (e.g. methanol or ethanol), and no solvent is present. In an alternative embodiment, HCl is generated in situ using e.g. acetyl chloride and formic acid; acetyl chloride and an alcohol (e.g. methanol or ethanol)l; or thionyl chloride and alcohol (e.g. methanol or ethanol), and water is present. In another embodiment, HCl is prepared in situ using acetyl chloride and formic acid, and no solvent is present. In an embodiment, HCl is prepared in situ using acetyl chloride and formic acid, and water is present.

### Example 1

### Coupling of Activated Meptazinol with Benzyl-4-Aminobenzoate

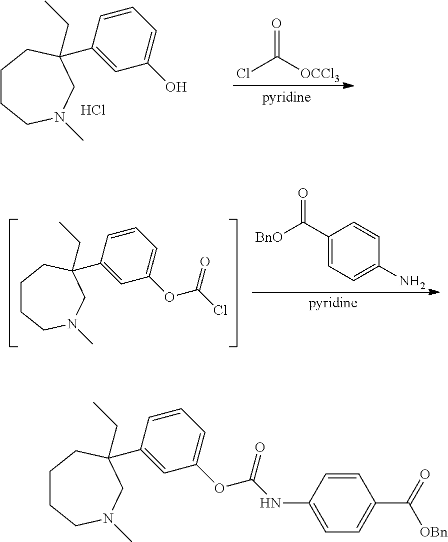

Meptazinol hydrochloride (187.5 g, 1.0 wt, 695 mmol), and dichloromethane (1875 mL, 10vol) were charged to a 5 L flange flask and cooled to −12 to −7° C. with mechanical stirring. Diphosgene (63.8 mL, 0.34vol, 0.77 eq) was charged followed by a line rinse with dichloromethane (94 mL, 0.5vol). Pyridine (141 mL, 0.75vol, 2.5 eq) was charged over a period of 80 minutes maintaining the temperature of the reaction at −12 to −7° C. followed by a line rinse with dichloromethane (94 mL, 0.5vol). The reaction was stirred at −12 to −7° C. for 17 hours when HPLC indicated >99% conversion (HPLC quenched with methanol, so observing the methyl carbonate).

Dichloromethane (938 mL, 5vol) was charged and the mixture warmed to 10 to 15° C. and concentrated to ca. 11vol under reduced pressure (at <15° C.). DCM (500 mL) was charged and the hazy solution and cooled to −2 to 3° C. Benzyl-4-aminobenzoate (150.0 g, 0.8 wt, 0.95 eq) was charged portionwise over 30 minutes maintaining −2 to 3° C. followed by a line rinse with dichloromethane (47 mL, 0.25vol). After stirring at −2 to 3° C. for 30 minutes, HPLC indicated that the chloroformate had been consumed (HPLC quenched with methanol, so observing disappearance of the methyl carbonate).

25% w/w aq KHCO₃ solution (562 mL, 3vol charged) and the resulting biphasic solution warmed to 18 to 23° C. The pH of the stirred mixture was adjusted to 7/8 with further 25% w/w aq KHCO₃ solution (750 mL, 4vol). The biphasic mixture was transferred to a seperatory funnel and the layers separated. The lower DCM layer was concentrated to dryness to give crude meptazinol PABA carbamate benzyl ester (342.8 g) as an oil.

The crude meptazinol PABA carbamate benzyl ester was purified by extensive chromatography; this gave a total of 118.5 g of material over 5 batches (Total yield: 35% of theoretical).

### Example 2

### Synthesis of the Acyl Imidazole Derivative of Meptazinol

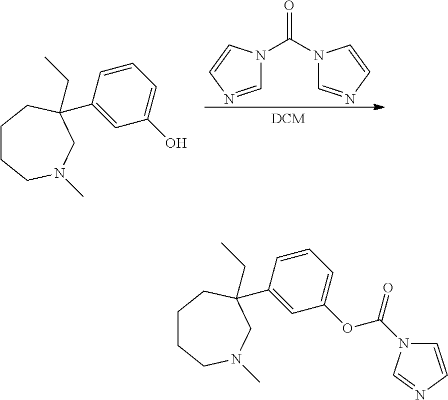

Meptazinol freebase (5.0 g) was slurried in DCM (10vol), and CDl (2 eq) was charged as a solid. After a 1 hour stir period at 18 to 23° C. ¹H NMR indicated complete conversion to the acyl imidazole adduct.

Water (5vol) was charged (exothermic and gas evolved), the layers were separated and the organic layer washed with further water (3×5vol). The organic layer was dried (sodium sulfate) and concentrated to dryness to give the acyl imidazole derivative as a viscous oil of 6.7 g (94.8% theoretical yield uncorrected) containing 2.8% w/w DCM.

### Example 3

### Coupling of the Acyl Imidazole Derivative of Meptazinol with Benzyl-4-Aminobenzoate

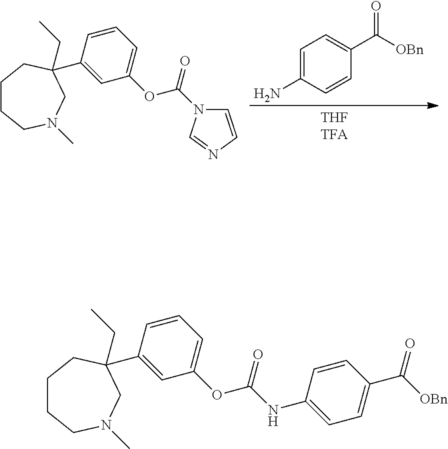

0.1 g of the acyl imidazole derivative of meptazinol and 1.2 eq of benzyl-4-aminobenzoate (hydrochloride salt) were suspended in 10 vol of THF, TFA (2 eq) was added and the mixture was heated at 35 to 40° C. for 20 hours. This provided meptazinol PABA carbamate with 79.1% conversion as determined by HPLC.

### Example 4

### Coupling of the Acyl Imidazole Derivative of Meptazinol with 4-Aminobenzoic Acid

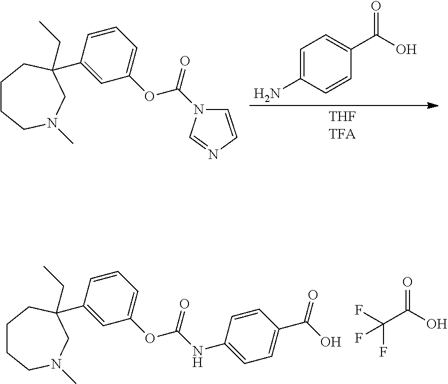

0.1 g of the acyl imidazole derivative of meptazinol and 1.2 eq 4-aminobenzoic acid (hydrochloride salt) were suspended in 10 vol of THF, TFA (2 eq) was added and the mixture was heated at 35 to 40° C. for 22 hours. This provided meptazinol PABA carbamate with 90.4% conversion as determined by HPLC.

### Example 5

### Synthesis of Meptazinol PABA Carbamate HCl

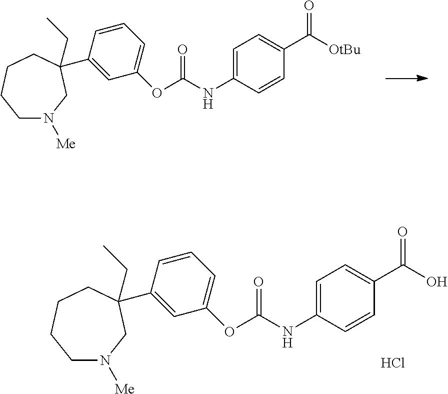

Meptazinol PABA carbamate t-butyl ester (1.0 g, 1 eq) and formic acid (14vol) were charged to the vessel. Water (0.06 mL, 1.5 eq) was charged, followed by acetyl chloride (0.20 mL, 1.3 eq). The reaction was stirred at 18-23° C. and monitored by LC-MS.

IPC by LC-MS after 1.5 h at 18-23° C. showed the reaction to be almost complete (<5% area MBC-TBE), further reaction time gave no change in reaction profile.

The reaction mixture was concentrated to dryness and azeotroped with toluene (3×10vol) and water (2×10vol) to remove residual formic acid which gave meptazinol PABA carbamate as a foam (0.7 g, 78.4% th).

### Example 6

### Preparation of Meptazinol Freebase

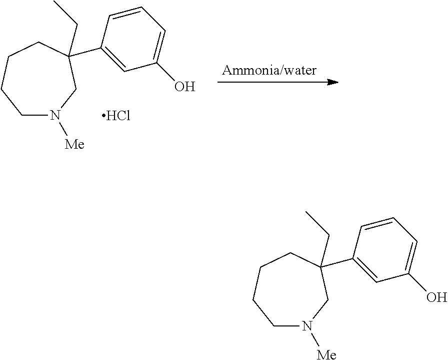

Meptazinol.HCl salt (5.0 g, 1 eq, 1 wt) was charged to a flask equipped with a magnetical stirrer bar. Water (8vol) was added and a clear solution was quickly obtained. The temperature was adjusted to 18-23° C. (pH solution=4.5). Aqueous ammonia was slowly charged and after 3 drops, the pH raised to 8.3 and a sticky solid appeared which blocked the stirrer. The magnetical stirrer was replaced by a mechanical stirrer. With a good agitation, addition of aqueous ammonia was resumed until pH 9 was reached (a total charge of 1.45 mL of aqueous ammonia used). The white slurry was stirred at 18-23° C. for 30 min and the pH rechecked. The white slurry was cooled to 5-10° C., stirred for 1 hr then filtered. The cake was washed with purified water (2×5vol) and dry on the filter under a flow N₂.

Performing the salt release of meptazinol hydrochloride under aqueous conditions using aq. ammonia as base gave a 92% th yield of meptazinol.

### Example 7

### Synthesis of Meptazinol PABA Carbamate from Meptazinol PABA Carbamate Benzyl Ester

Meptazinol PABA carbamate benzyl ester (1.00 wt, 1.00 eq), THF (19.6 wt, 22.0vol) and palladium on charcoal (5% dry powder type 87G, 0.05 wt) were charged to a vessel. The mixture was stirred and the temperature maintained at 18-23° C. The vessel was purged three times by vacuum/argon purge cycles at 18-23° C. The reaction vessel was then heated to 38-43° C. and stirred for 4 hours under hydrogen at atmospheric pressure until complete by HPLC analysis, (pass criterion ≦0.2% area meptazinol PABA carbamate benzyl ester). The vessel was purged three times by vacuum/argon purge cycles at 38-43° C. The reaction mixture was filtered at 38-43° C. under argon to 69 to 78% w/w.

Patents, patent applications, and non-patent literature cited in herein are hereby incorporated by reference in their entirety.

1. A method for synthesizing an opioid prodrug of formula (I) or a pharmaceutically acceptable salt thereof: 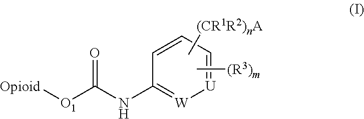
wherein:
“Opioid-O₁” is an opioid drug fragment having a phenolic hydroxyl residue and O₁ is said phenolic hydroxyl residue of the opioid;
W and U are each independently selected from the group consisting of: —CR⁴═ and —N═;
R¹ and R² are each independently selected from the group consisting of: H, hydroxy, carboxy, carboxamido, imino, alkanoyl, cyano, cyanomethyl, nitro, amino, halogen, C₁₋₆ alkyl, C₁₋₆ haloalkyl, C₁₋₆ alkoxy, O₁₋₆ haloalkoxy, C₃₋₆ cycloalkyl, aryl, aryl-C₁₋₆ alkyl and C₁₋₆ alkyl aryl;
n is 0, 1 or 2;
m is 0, 1 or 2;
R³ is independently selected from the group consisting of: hydroxy, carboxy, carboxamido, imino, alkanoyl, cyano, cyanomethyl, nitro, amino, halogen, C₁₋₆ alkyl, C₁₋₆ alkoxy, C₁₋₆ haloalkoxy, C₃₋₆ cycloalkyl, aryl, aryl-C₁₋₆ alkyl and O₁₋₆ alkyl aryl;
R⁴ is H or R³;
A is a carboxylic acid group (i.e. —CO₂H) or is a protected carboxylic acid group;
the method including the step of:
i) treating an opioid (i.e. a compound of Formula opioid-O₁—H), in the form of a salt or a freebase, with a carbonyl synthon of Formula (II), 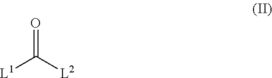
to form an activated intermediate of Formula (III) 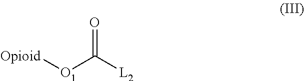
wherein:
L¹ and L² are each independently a leaving group;
the method further including the step of:
ii) reacting the activated intermediate of Formula III with an amine of Formula IV, in the form of a salt or a freebase, 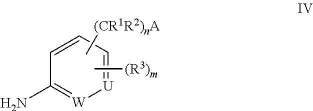
to provide the opioid prodrug of formula I.

2. The method of claim 1, wherein L₁ and L₂ are imidazole.

3. The method of claim 1, wherein L¹ and L² are independently selected from halo and trihalomethyloxy.

4. The method of claim 1, wherein the opioid drug having a phenolic hydroxyl group is an opioid drug selected from the group consisting of: hydromorphone, butorphanol, buprenorphine, dezocine, dextrorphan, hydroxyopethidine, ketobemidone, levorphanol, meptazinol, morphine, nalbuphine, oxymorphone, pentazocine, tapentadol, dihydroetorphine, diprenorphine, etorphine, nalmefene, oripavine, phenazocine, O-desmethyl tramadol, ciramadol, levallorphan, tonazocine, eptazocine and a phenolically hydroxylated, e.g. a 2-, 3- or 4-phenolically hydroxylated phenazepine analgesic, such as a 2-, 3- or 4-phenolically hydroxylated ethoheptazine, proheptazine, metethoheptazine, metheptazine, and any other analgesic.

5. The method of claim 4, wherein the opioid is meptazinol or buprenorphine.

6. The method of claim 1, wherein A is a carboxylic acid group or a protected carboxylic acid group selected from the group consisting of: —CO₂R⁵; —CN; —C(OR^{a})₃; —C(O)(SR⁵); and 2-oxazalinyl;
wherein R₅ is H or a protecting group;
wherein the 2-oxazalinyl group is optionally substituted with 1 or 2 substituents selected from the group consisting of: C₁-C₄ alkyl, benzyl (optionally substituted with one or two substituents selected from C₁-C₄ alkyl, C₁-C₄ alkoxy, C₁-C₄haloalkyl, C₁-C₄ haloalkoxy, halogen, CN and NO₂) and C₁-C₄ haloalkyl; and
wherein R^{a} is independently at each occurrence selected from the group consisting of: C₁-C₄ alkyl and benzyl.

7. The method of claim 1, wherein U is —CH═ and W is —CH═, n is 0 and m is 0.

8. The method of claim 1, wherein the opioid prodrug of formula I has the structure:

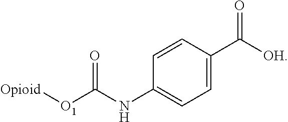

9. The method of claim 1, wherein the method further comprises a step prior to reacting the opioid with a carbonyl synthon of Formula II including treating a salt of the opioid with a base to form the opioid freebase.

10. The method of claim 1, wherein Step i) is performed in the presence of a base.

11. The method of claim 10, wherein the base is an organic base.

12. The method of claim 10, wherein the base is an inorganic base.

13. The method of claim 1, wherein Step i) is not performed in the presence of a base.

14. The method of claim 1, wherein Step i) is performed in a non-nucleophillic solvent.

15. The method of claim 1, wherein Step i) is performed at a temperature of from about −50° C. to about 70° C.

16. The method of claim 1, wherein Step ii) is performed in the presence of a base.

17. The method of claim 16, wherein the base is an organic base.

18. The method of claim 16, wherein the base is an inorganic base.

19. The method of claim 1, wherein Step ii) is performed in the presence of an acid.

20. The method of claim 1, wherein Step ii) is performed in a solvent selected from the group consisting of DCM, toluene, chlorobenzene, benzene, diethyl ether, ethyl acetate, MeCN, acetic acid, formic acid, NMP, DMF, THF, TBME, DMSO, dioxane, pyridine, hexane, dichloroethane, xylene and acetonitrile or a combination of two or more of said solvents.

21. The method of claim 1, wherein Step ii) is performed at a temperature of from about −50° C. to about 80° C.

22. The method of claim 1, wherein A is a protected carboxylic acid group of the formula —CO₂R₅ (wherein R⁵ is not H) and the method further includes the subsequent step of removing the protecting group R⁵ from the opioid prodrug of formula I to obtain an opioid prodrug of formula I in which R⁵ is H.

23. A method for synthesizing an activated opioid intermediate of Formula III: 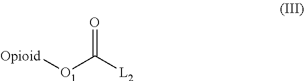
the method including the step of treating an opioid (i.e. a compound of Formula opioid-O₁—H), in the form of a salt or a freebase, with a carbonyl synthon of Formula (II), 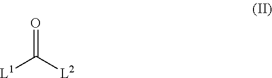
to form an activated intermediate of Formula (III) 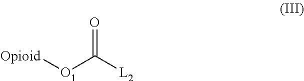
wherein:
“Opioid-O₁” is an opioid drug fragment having a phenolic hydroxyl residue and O₁ is said phenolic hydroxyl residue of the opioid;
wherein:
L¹ and L² are each independently a leaving group.

24. (canceled)

25. The method of claim 11, wherein the base is selected from the group consisting of pyridine; pyridine substituted with one or two substituents selected from C₁-C₄ alkyl, C₁-C₄ alkoxy, C₁-C₄haloalkyl, C₁-C₄ haloalkoxy, halogen, CN and NO₂; imidazole; imidazole substituted with one or two substituents selected from C₁-C₄ alkyl, C₁-C₄ alkoxy, C₁-C₄haloalkyl, C₁-C₄ haloalkoxy, halogen, CN and NO₂; a trialkylamine; a trialkylamine wherein two of the alkyl groups form a 5-7 membered saturated ring, and mixtures thereof.

26. The method of claim 12, wherein the base is selected from the group consisting of a hydroxide, a carbonate, a bicarbonate of a Group 1 or Group 2 metal, and mixtures thereof.

27. The method of claim 14, wherein the solvent is selected from the group consisting of hexane, heptane, cyclohexane, DCM, dichloroethane, benzene, toluene, chlorobenzene and mixtures thereof.

28. The method of claim 17, wherein the base is selected from the group consisting of: pyridine; pyridine substituted with one or two substituents selected from C₁-C₄ alkyl, C₁-C₄ alkoxy, C₁-C₄haloalkyl, C₁-C₄ haloalkoxy, halogen, CN and NO₂; imidazole; imidazole substituted with one or two substituents selected from C₁-C₄ alkyl, C₁-C₄ alkoxy, C₁-C₄haloalkyl, C₁-C₄ haloalkoxy, halogen, CN and NO₂; a trialkylamine; a trialkylamine wherein two of the alkyl groups form a 5-7 membered saturated ring, and mixtures thereof.

29. The method of claim 18, wherein the base is selected from the group consisting of: a hydroxide, a carbonate, a bicarbonate of a Group 1 or Group 2 metal, and mixtures thereof.

30. The method of claim 19, wherein the acid is selected from the group consisting of: HCl, TFA, acetic acid, formic acid, propionic acid, pyridinium para-toluene sulphonate, para-toluene sulfonic acid, camphor sulfonic acid, and mixtures thereof.
